(19) United States
(12) Patent Application Publication
(10) Pub. No.: US 2009/0327515 A1
(43) Pub. Date: Dec. 31, 2009

(54) Medical Monitor With Network Connectivity

(76) Inventor: Thomas Price, Lakewood Jefferson County, CO (US)

Correspondence Address: NELLCOR PURITAN BENNETT LLC;ATTN: IP LEGAL, 6135 Gunbarrel Avenue, Boulder, CO, 80301, US

(21) Appl. No.: 12/165,112

(22) Filed: Jun. 30, 2008

(51) Int. Cl. G06F 15/16; A61B 5/02

(52) U.S. Cl. 709/236; 600/324

(57) ABSTRACT

The present disclosure provides for the use of physiological monitors capable of communicating over a network. In one embodiment, the physiological monitors may utilize a network layer protocol having an address space for each packet that is greater than 32 bits in length. In one such embodiment, address exhaustion on a network may be addressed by using addresses greater than 32 bits in length at the network layer.

### BACKGROUND

The present disclosure relates generally to medical devices, and, more particularly, to a physiological monitor for use on a network.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light and not as admissions of prior art.

In the field of healthcare, caregivers (e.g., doctors and other healthcare professionals) often desire to monitor certain physiological characteristics of their patients. Accordingly, a wide variety of monitoring devices have been developed for monitoring many such physiological characteristics. These monitoring devices often provide doctors and other healthcare personnel with information that facilitates provision of the best possible healthcare for their patients. As a result, such monitoring devices have become a perennial feature of modern medicine.

One technique for monitoring physiological characteristics of a patient is commonly referred to as pulse oximetry, and the devices built based upon pulse oximetry techniques are commonly referred to as pulse oximeters. Pulse oximeters may be used to measure and monitor various blood flow characteristics of a patient. For example, a pulse oximeter may be utilized to monitor the blood oxygen saturation of hemoglobin in arterial blood, the volume of individual blood pulsations supplying the tissue, and/or the rate of blood pulsations corresponding to each heartbeat of a patient. In practice, a pulse oximeter may be deployed in proximity to a patient, such as beside the patient's bed. However, it may be desirable to access data or measurements acquired by the pulse oximeter from a remote location.

### BRIEF DESCRIPTION OF THE DRAWINGS

Advantages of the disclosure may become apparent upon reading the following detailed description and upon reference to the drawings in which:

FIG. 1 is a perspective view of a pulse oximeter coupled to a multi-parameter patient monitor and a sensor in accordance with embodiments;

FIG. 2 is a block diagram of the pulse oximeter and sensor coupled to a patient in accordance one embodiment;

FIG. 3 is a block diagram of the pulse oximeter and sensor coupled to a patient in accordance another embodiment; and

FIG. 5 is a block diagram of a network configuration in accordance with embodiments.

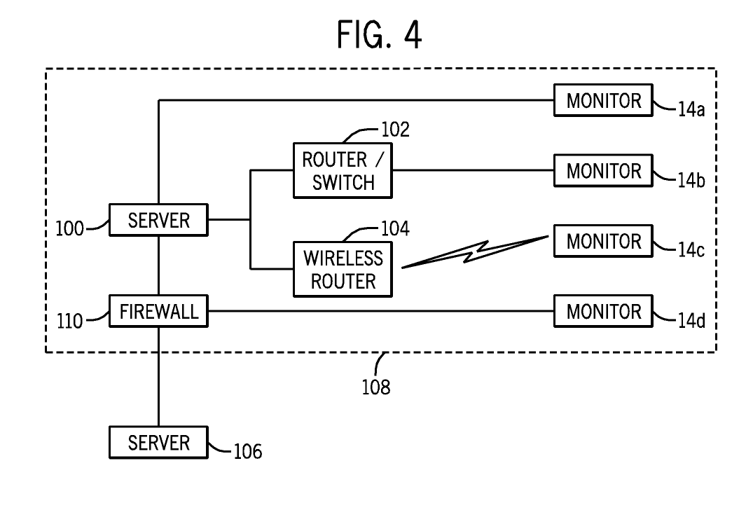

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Physiological monitors, such as pulse oximeters may be employed to monitor one or more physiological characteristics of a patient. Typically the physiological monitor is provided at the bedside of the patient or in similar close proximity. However, it may be desirable to monitor the patient from a remote location, such as a nurse's station or doctor's office. Therefore, it may be desirable to provide the physiological monitor with some form of network connectivity to allow communication to and from the physiological monitor from another location on the network. In some implementations, such network connectivity may be accomplished using wired or wireless mechanisms. Further, to avoid address exhaustion, a network protocol may be supported by the physiological monitor that allows the use of large address spaces, such as address spaces that are 128 bits long or longer. An example, of such a network protocol is Internet Protocol version 6 (IPv6).

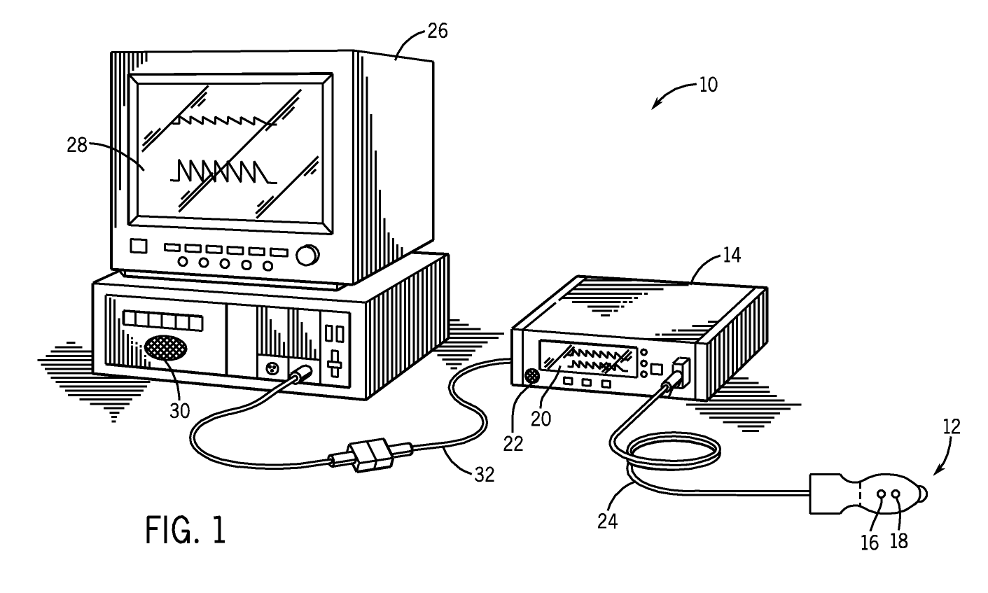

FIG. 1 is a perspective view of such a pulse oximetry system 10 in accordance with an embodiment. The system 10 includes a sensor 12 and a pulse oximetry monitor 14. The sensor 12 includes an emitter 16 for emitting light at certain wavelengths into a patient's tissue and a detector 18 for detecting the light after it is reflected and/or absorbed by the patient's tissue. The monitor 14 may be capable of calculating physiological characteristics received from the sensor 12 relating to light emission and detection. Further, the monitor 14 includes a display 20 capable of displaying the physiological characteristics, other information about the system, and/or alarm indications. The monitor 14 also includes a speaker 22 to provide an audible alarm in the event that the patient's physiological characteristics exceed a threshold. The sensor 12 may be communicatively coupled to the monitor 14 via a cable 24. However, in other embodiments a wireless transmission device or the like may be utilized instead of or in addition to the cable 24.

In the illustrated embodiment, the pulse oximetry system 10 also includes a multi-parameter patient monitor 26. In addition to the monitor 14, or alternatively, the multi-parameter patient monitor 26 may be capable of calculating physiological characteristics and providing a central display 28 for information from the monitor 14 and from other medical monitoring devices or systems. For example, the multi-parameter patient monitor 26 may display a patient's SpO₂ and pulse rate information from the monitor 14 and blood pressure from a blood pressure monitor on the display 28. Additionally, the multi-parameter patient monitor 26 may indicate an alarm condition via the display 28 and/or a speaker 30 if the patient's physiological characteristics are found to be outside of the normal range. The monitor 14 may be communicatively coupled to the multi-parameter patient monitor 26 via a cable 32 coupled to a sensor input port or a digital communications port. In addition, the monitor 14 and/or the multi-parameter patient monitor 26 may be connected to a network, as discussed herein, to enable the sharing of information with servers or other workstations.

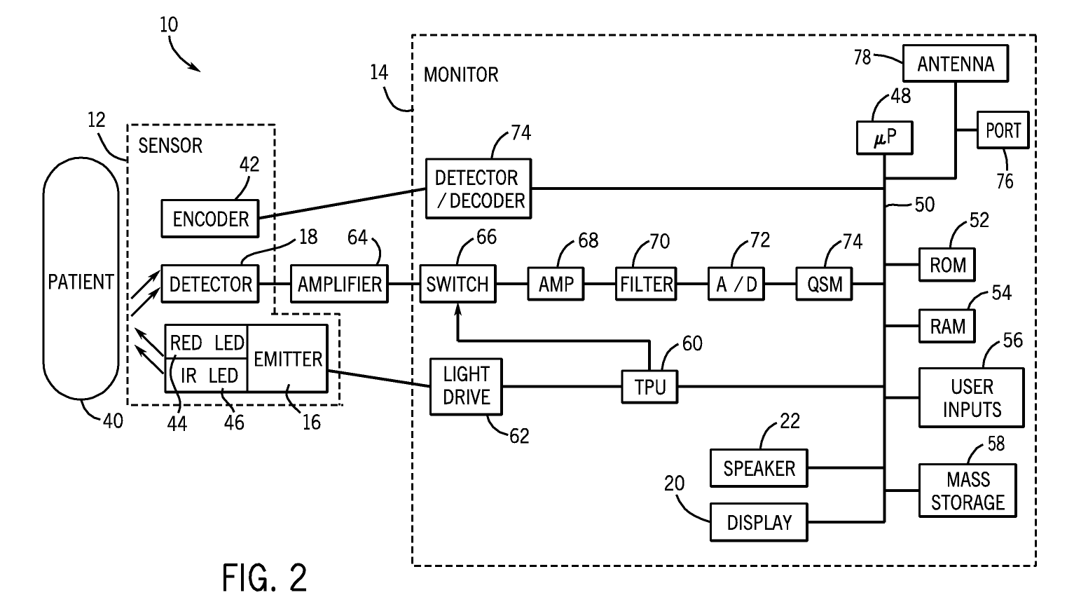

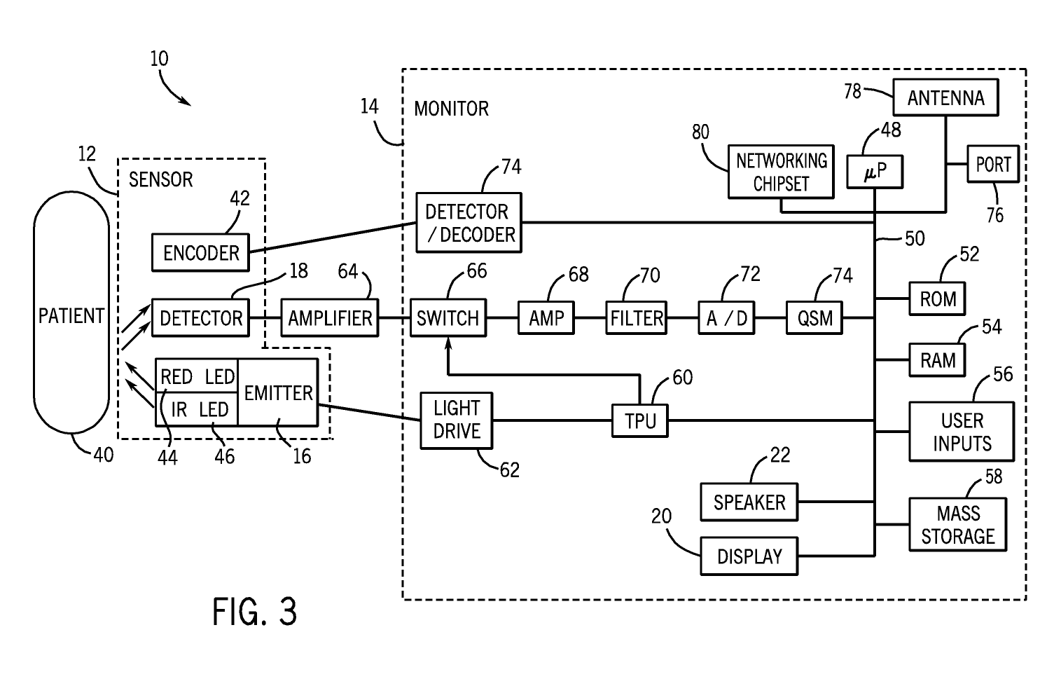

FIGS. 2 and 3 are block diagrams of exemplary pulse oximetry systems 10 of FIG. 1 coupled to a patient 40 in accordance with present embodiments. Examples of pulse oximeters that may be used in the implementation of the present disclosure include pulse oximeters available from Nellcor Puritan Bennett LLC, but the following discussion may be applied to other pulse oximeters and medical devices. Specifically, certain components of the sensor 12 and the monitor 14 are illustrated in FIG. 2. The sensor 12 may include the emitter 16, the detector 18, and an encoder 42. It should be noted that the emitter 16 may be capable of emitting at least two wavelengths of light, e.g., RED and IR, into a patient's tissue 40. Hence, the emitter 16 may include a RED LED 44 and an IR LED 46 for emitting light into the patient's tissue 40 at the wavelengths used to calculate the patient's physiological characteristics. In certain embodiments, the RED wavelength may be between about 600 nm and about 700 nm, and the IR wavelength may be between about 800 nm and about 1000 nm. Alternative light sources may be used in other embodiments. For example, a single wide-spectrum light source may be used, and the detector 18 may be capable of detecting certain wavelengths of light. In another example, the detector 18 may detect a wide spectrum of wavelengths of light, and the monitor 14 may process only those wavelengths which are of interest. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In one embodiment, the detector 18 may be capable of detecting the intensity of light at the RED and IR wavelengths. In operation, light enters the detector 18 after passing through the patient's tissue 40. The detector 18 may convert the intensity of the received light into an electrical signal. The light intensity may be directly related to the absorbance and/or reflectance of light in the tissue 40. That is, when more light at a certain wavelength is absorbed or reflected, less light of that wavelength is typically received from the tissue by the detector 18. After converting the received light to an electrical signal, the detector 18 may send the signal to the monitor 14, where physiological characteristics may be calculated based at least in part on the absorption of the RED and IR wavelengths in the patient's tissue 40.

The encoder 42 may contain information about the sensor 12, such as what type of sensor it is (e.g., whether the sensor is intended for placement on a forehead or digit) and the wavelengths of light emitted by the emitter 16. This information may allow the monitor 14 to select appropriate algorithms and/or calibration coefficients for calculating the patient's physiological characteristics. The encoder 42 may, for instance, be a coded resistor which stores values corresponding to the type of the sensor 12 and/or the wavelengths of light emitted by the emitter 16. These coded values may be communicated to the monitor 14, which determines how to calculate the patient's physiological characteristics. In another embodiment, the encoder 42 may be a memory on which one or more of the following information may be stored for communication to the monitor 14: the type of the sensor 12; the wavelengths of light emitted by the emitter 16; and the propel calibration coefficients and/or algorithms to be used for calculating the patient's physiological characteristics. Pulse oximetry sensors capable of cooperating with pulse oximetry monitors include the OxiMax® sensors available from Nellcor Puritan Bennett LLC.

Signals from the detector 18 and the encoder 42 may be transmitted to the monitor 14. The monitor 14 generally may include one or more processors 48 connected to an internal bus 50. Also connected to the bus may be a read-only memory (ROM) 52, a random access memory (RAM) 54, user inputs 56, one or more mass storage devices 58 (such as hard drives, disk drives, or other magnetic, optical, and/or solid state storage devices), the display 20, or the speaker 22. A time processing unit (TPU) 60 may provide timing control signals to a light drive circuitry 62 which controls when the emitter 16 is illuminated and the multiplexed timing for the RED LED 44 and the IR LED 46. The TPU 60 control the gating-in of signals from detector 18 through an amplifier 64 and a switching circuit 66. These signals may be sampled at the proper time, depending upon which light source is illuminated. The received signal from the detector 18 may be passed through an amplifier 68, a low pass filter 70, and an analog-to-digital converter 72. The digital data may then be stored in a queued serial module (QSM) 74 for later downloading to the RAM 54 or mass storage 58 as the QSM 74 fills up. In one embodiment, there may be multiple separate parallel paths having the amplifier 68, the filter 70, and the A/D converter 72 for multiple light wavelengths or spectra received.

Signals corresponding to information about the sensor 12 may be transmitted from the encoder 42 to a decoder 74. The decoder 74 may translate these signals to enable the microprocessor to determine the proper method for calculating the patient's physiological characteristics, for example, based generally on algorithms or look-up tables stored in the ROM 52 or mass storage 58. In addition, or alternatively, the encoder 42 may contain the algorithms or look-up tables for calculating the patient's physiological characteristics.

The monitor 14 may also include one or more features to facilitate communication with other devices in a network environment. For example, the monitor 14 may include a network port 76 (such as an Ethernet port) and/or an antenna 78 by which signals may be exchanged between the monitor 14 and other devices on a network, such as servers, routers, switches, workstations and so forth. As depicted in FIG. 3, in some embodiments, such network functionality may be facilitated by the inclusion of a networking chipset 80 within the monitor 14, though in other embodiments the network functionality may instead be provided by the processor(s) 48.

In embodiments where network functionality is provided on the monitor 14, the monitor may support one or more different network communication protocols. For example, in one embodiment the monitor 14 may support a multi-layer network communication model using Transmission Control Protocol (TCP) as the transport layer and Internet Protocol (IP) as the network layer. In such embodiments, the respective code and/or instructions supporting the various protocols may be implemented as hardware, software, and/or firmware on a networking chipset 80. In another embodiment, the respective code and/or instructions supporting the various protocols may be executed by the processor(s) 48 and stored as firmware in the ROM 52 or as software on the mass storage device 58.

Due to the number of devices that may be members of a network in a hospital or clinical environment, it may be desirable to implement network communication protocols that provide an extensive address space. For example, Internet Protocol version 6 (IPv6) provides for 128 bit addresses (as opposed to 32 bit addresses in IPv4) for data packets generated in conformity with the protocol. The lengthier address space associated with IPv6 relative to previous versions of IP may allow for a sufficient number of addresses to exist on the network so that subnets, submasks, and/or network address translation (NAT) do not need to be employed to provide unique addresses for each device on the network.

Therefore, in some embodiments where the number of available addresses may be an issue, the monitor 14 may be capable of storing, executing, or otherwise implementing a communication layer, such as a network layer of a multi-layer network model, capable of supporting extended address spaces, such as 128 bit (or greater) addresses. For example, in one embodiment, a physiological monitor 14, such as a pulse oximeter, may implement an extended address space network layer, such as IPv6 or other network layer protocols using addresses greater than 32 bits in length, i.e., 128 bits, 256 bits, and so forth. Thus, in such an embodiment, packets generated in compliance with the network layer protocol include a header that is greater than 32 bits in length. In such an embodiment, the monitor 14 may also support other communication layers that interact with the network layer, such as a transport layer and a data link layer. For example, in one embodiment the monitor 14 may be capable of implementing TCP, User Datagram Protocol (UDP), or another suitable transport layer and may be capable of implementing 802.11, 802.16, Wi-Fi, token ring, Ethernet, fiber distributed data interface (FDDI), or another suitable data link layer. In one such embodiment, the network on which the monitor 14 resides may operate without utilizing subnets, submasks, and/or NAT.

With the foregoing in mind, various network configurations for a networkable monitor 14 are depicted in FIG. 4. For example, in one network configuration the monitor 14a and monitor 14b may communicate with a server 100 via a wire connection, either directly or via a router or switch 102, respectively. Similarly, in network configurations supporting wireless protocols, a monitor 14c may communicate with a server 100 via a wireless router 104 or other wireless communication device. In another configuration, a monitor 14d may communicate with an external server 106 located outside a hospital network or other local network 108. In configurations where the monitor 14 communicates with devices outside the local network 108, communication may pass through a firewall 110 or other security device regulating inter-network communications.

While only certain features have been illustrated and described herein, many modifications and changes will occur to those skilled in the art. It is, therefore, to be understood that the appended claims are intended to cover all such modifications and changes as fall within their true spirit.

What is claimed is:

1. A physiological monitor comprising:
a network port or an antenna; and
a processor capable of at least communicating with other devices on a network via the network port or the antenna, wherein the processor is capable of at least communicating using a network layer protocol that utilizes addresses that are greater than 32 bits in length.

2. The physiological monitor of claim 1, wherein the network layer protocol utilizes 128 bit addresses.

3. The physiological monitor of claim 1, wherein the network layer protocol comprises Internet Protocol version 6.

4. The physiological monitor of claim 1, wherein the physiological monitor comprises a pulse oximeter.

5. A physiological monitor comprising:
a network port or an antenna;
a processor capable of at least communicating with other devices on a network via the network port or the antenna; and
a networking chipset capable of at least implementing a network layer protocol that utilizes addresses that are greater than 32 bits in length to facilitate the communication between the processor and the network.

6. The physiological monitor of claim 5, wherein the network layer protocol utilizes 128 bit addresses.

7. The physiological monitor of claim 5, wherein the network layer protocol comprises Internet Protocol version 6.

8. The physiological monitor of claim 5, wherein the physiological monitor comprises a pulse oximeter.

9. A method of transmitting data between a monitor and a network,
comprising:
utilizing a network layer protocol to handle data packets having addresses that are greater than 32 bits in length; and
transmitting and receiving data packets generated in accordance with the network layer protocol.

10. The method of claim 9, wherein the network layer protocol utilizes 128 bit addresses.

11. The method of claim 9, wherein the network layer protocol comprises Internet Protocol version 6.

12. The method of claim 9, wherein the act of transmitting and receiving data packets comprises transmitting and receiving data packets over a wireless network connection.

13. The method of claim 9, wherein the act of transmitting and receiving data packets comprises transmitting and receiving data packets over an Ethernet network.

14. The method of claim 9, comprising utilizing at least one of Transmission Control Protocol (TCP) or User Datagram Protocol (UDP) as a transport layer protocol for transmitting and receiving the data packets.

15. The method of claim 9, comprising utilizing at least one of a 802.11 protocol, a 802.16 protocol, a Wi-Fi protocol, a token ring protocol, an Ethernet protocol, or a fiber distributed data interface (FDDI) protocol as a data link layer protocol for transmitting and receiving the data packets.

16. A system, comprising:
one or more networks;
one or more monitors capable of at least communicating across the one or more networks utilizing a network layer protocol that employs an address space for data packets that is greater than 32 bits in length; and
one or more additional devices capable of at least communicating with the one or more monitors using the network layer protocol.

17. The system of claim 16, wherein the one or more networks do not utilize one or more of subnets, submasks, or network address translation.

18. The system of claim 16, wherein the one or more monitors comprise pulse oximeter monitors.

19. The system of claim 16, wherein the network layer protocol comprises Internet Protocol version 6.

20. The system of claim 16, wherein the address space for the data packets is 128 bits long.

21. A pulse oximeter, comprising:
at least one of a network port or an antenna capable of at least exchanging data packets over a network; and
a processor or a networking chipset capable of at least formatting the data packets to each have an address greater than 32 bits in length in accordance with a network layer protocol.

22. The pulse oximeter of claim 21, wherein the data packets each have an address that is 128 bits long.

23. The pulse oximeter of claim 21, wherein the network layer protocol comprises Internet Protocol version 6.

24. The pulse oximeter of claim 21, wherein the processor or networking chipset is also capable of at least formatting the data packets in accordance with at least one of Transmission Control Protocol (TCP) or User Datagram Protocol (UDP) as a transport layer.

25. The pulse oximeter of claim 21, wherein the processor or networking chipset is also capable of at least formatting the data packets in accordance with at least one of a 802.11 protocol, a 802.16 protocol, a Wi-Fi protocol, a token ring protocol, an Ethernet protocol, or a fiber distributed data interface (FDDI) protocol as a data link layer protocol.
